(19) United States
(12) Patent Application Publication
(10) Pub. No.: US 2025/0164593 A1
(43) Pub. Date: May 22, 2025

(54) WIRELESS PHYSIOLOGICAL GATING VIA REFLECTOMETRY AND SECONDARY DEVICE

(71) Applicant: GE Precision Healthcare LLC, Waukesha, WI (US)

(72) Inventors: Randall Henry Buchwald, Waukesha, WI (US); Andrew Thomas Krill, Milwaukee, WI (US)

(21) Appl. No.: 18/518,297

(22) Filed: Nov. 22, 2023

(51) Int. Cl. G01R 33/567; A61B 5/055

(52) U.S. Cl. CPC G01R 33/5673; A61B 5/055

(57) ABSTRACT

Wirelessly transferring data (e.g., an electrocardiogram (ECG) waveform) from a secondary device (e.g., an ECG device) to a primary device (e.g., an MRI machine) where the antenna of the primary device is configured to wirelessly receive an analog physiological signal of interest from the secondary device, where the antenna of the primary device is coupled in near-field with a resonant antenna of the secondary device, and the secondary device includes circuitry to vary an impedance of or vary a tuning of the resonant antenna of the secondary device so as to influence an electromagnetic environment and a tuning of the antenna of the primary device, the change in electromagnetic environment being detected by the primary device and corresponding to the analog physiological signal of interest from the secondary device.

### TECHNICAL FIELD

The present disclosure relates to wirelessly transferring analog physiological data from a secondary device to a primary device, and, more particularly, for wirelessly transferring data such as electrocardiogram (ECG) waveform data from a secondary device to a magnetic resonance imaging (MRI).

### BACKGROUND

Magnetic resonance imaging (MRI) may be used to obtain internal physiological information of a patient, including for cardiac imaging and imaging other tissues within a patient's body. In certain areas, such as portions in the torso, it is typically desirable to obtain an image at a particular point in a variable cycle (e.g. respiratory cycle and/or cardiac cycle), such as a peak of the variable cycle, to analyze behavior during that peak. Physiological gating is an option for characterizing different attributes of an organ for imaging.

Common techniques of gating may include cardiac, respiratory, and peripheral pulse gating, and such techniques of gating have uses in numerous medical applications across diagnostic modalities such as CT, MRI, x-ray, ultrasound, and position emission tomography (PET). Respiratory gating and cardiac gating, for example, are important for cardiac imaging while using imaging modalities such as CT and MRI to minimize motion-related artifacts resulting from motion due to the patient's respiration and the patient's heartbeat.

Accordingly, respiratory gating and/or cardiac gating are often used in acquisition of MRI data, which rely on detection of a particular point in the motion cycle as a trigger to repeatedly acquire data at approximately the same phase of the motion cycle. Sensor systems are utilized to sense respiration activity and cardiac potentials. Respiratory monitors utilizing bellows sensors are typically used for detecting respiratory wave forms, which detect chest expansion utilizing a belt and bellows comprising a pressure sensor. An electrocardiogram (ECG) device with electrodes attached to the patient is generally utilized to monitor the cardiac cycle. However, such monitoring methods typically require wired/cable electrical connections with the MRI machine and add time for preparation of the patient and scanning system setup.

### BRIEF DESCRIPTION

In one embodiment, a method of wireless physiological gating in a magnetic resonance imaging (MRI) machine includes wirelessly receiving an analog physiological signal of interest from a secondary device and a primary device, the primary device comprising an antenna of the MRI machine being coupled in near-field with a resonant antenna of the secondary device, and the secondary device having circuitry configured to vary an impedance of or otherwise vary a tuning of the resonant antenna of the secondary device so as to influence an electromagnetic environment or tuning of the antenna of the MRI machine, the change in electromagnetic environment being detected by the primary device and corresponding to the analog physiological signal of interest from the secondary device.

In one embodiment, a system configured to wirelessly transfer data from a secondary device to a primary device comprises circuitry and an antenna/primary coil of the primary device configured to wirelessly receive an analog physiological signal of interest from the secondary device, the antenna of the primary device being coupled in near-field with a resonant antenna of the secondary device, and the secondary device having circuitry configured to vary an impedance of or otherwise vary a tuning of the resonant antenna of the secondary device so as to influence an electromagnetic environment or tuning of the antenna of the primary device, the change in electromagnetic environment being detected by the circuitry within the primary device and corresponding to the analog physiological signal of interest from the secondary device.

In one embodiment, a system to wirelessly transfer data from an electrocardiogram (ECG) device to a magnetic resonance imaging (MRI) machine comprises circuitry and an antenna/primary coil of the MRI machine configured to wirelessly receive an analog physiological signal of interest from the ECG device, the antenna of the MRI machine being coupled in near-field with a resonant antenna of the ECG device, and the ECG device having circuitry configured to vary an impedance of or otherwise vary a tuning of the resonant antenna of the ECG device so as to influence an electromagnetic environment or a tuning of the antenna of the MRI machine, the change electromagnetic environment being detected by the circuitry within the MRI machine and corresponding to the analog physiological signal of interest from the ECG device, wherein the MRI machine comprises circuitry and one or more antenna configured to probe the electromagnetic environment and measure changes in the electromagnetic environment corresponding to the analog physiological signal of interest from the ECG device. In one aspect, the analog physiological signal of interest comprises an electrical QRS signal or electrocardiogram (ECG) waveform, and the ECG device comprises electrode connections to a patient and a resonant circuit configured to directly modulate an impedance of or otherwise directly modulate the tuning of the resonant antenna of the secondary device so as to correlate to the ECG waveform. In another aspect, the ECG device is free from electrical interconnection with the MRI machine and is configured so as to permit wireless data transfer of the analog physiological signal of interest from the ECG device to the MRI machine without the ECG device using a radio frequency (RF) transmitter.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure is described with reference to the schematic diagrams, depictions, circuit diagrams, exemplary signal and reflection coefficient shapes and plots, and illustrations in the following figures.

FIG. 1 is a schematic diagram of an exemplary magnetic resonance imaging (MRI) system with an MRI machine and a secondary device, according to embodiments.

FIG. 2 is an illustration of an exemplary table of an MRI system/machine, according to embodiments.

FIG. 3 depicts an exemplary primary coil/antenna and matching circuitry, or sensor, of an MRI system/machine, according to embodiments.

FIG. 4 is a circuit diagram of an exemplary coupling circuit and matching circuitry of an MRI system/machine, according to embodiments.

FIG. 5 is a circuit diagram of FIG. 4 with modifications to provide an exemplary resonant antenna and resonant circuit with variable impedance/variable antenna tuning element(s) of a secondary device, according to embodiments.

FIG. 6 is a schematic diagram of a primary coil/antenna and reflectometer circuit within a primary device coupled in near-field to an exemplary secondary device, according to embodiments.

FIG. 7 is a circuit diagram of an exemplary resonant antenna and resonant circuit of a secondary device, according to embodiments.

FIG. 8A is a schematic diagram of an MRI system having a self-resonant spiral or similar antenna and a reflectometer circuit of the MRI machine coupled in near-field with a resonant antenna and resonant circuit of a secondary device, according to embodiments.

FIG. 8B is a schematic diagram of an MRI system configured to measure a transmission path between a transmit antenna and a receive antenna of the MRI machine, the with a resonant antenna of the secondary device coupled in near-field one or both the transmit and receive antenna, according to embodiments.

FIG. 9 depicts a shape of an exemplary analog physiological signal of interest, according to embodiments.

FIG. 10 depicts a plot of changes in a reflection coefficient (S11) for an exemplary primary coil/antenna and reflectometer circuit, according to embodiments.

### DETAILED DESCRIPTION

The present disclosure describes embodiments for wirelessly receiving an analog physiological signal from a secondary device via circuitry within an MRI machine, where the analog physiological signal may comprise, for example, an ECG signal that may be used for gating in the MRI machine.

The present inventors have recognized the desirability for non-contact physiological gating methods in order to avoid the patient preparation and set up time required for patient-contact based methods requiring, for example, patient preparation to attach air pressure bellows, finger (pulse) sensors, or wired ECG cables. Other techniques, such as the non-contact-respiratory (NCR) technique described and taught in the inventors U.S. patent Ser. No. 11/419,516 B2, which is incorporated by reference herein for all purposes, do not provide a true electrical ECG waveform with its minimal trigger delay. Existing wireless ECG gating solutions may provide an electrical QRS signal, however such solutions (which utilize an RF transmitter and communications via Bluetooth or Wi-Fi) are expensive, have limited bandwidth (with process and datalink delays), and have limited capabilities to handle gradient noise.

The aforementioned NCR technique (described in U.S. patent Ser. No. 11/419,516 B2, as incorporated by reference above), operates in a unique manner, measuring the impedance changes in the bore due to respiratory or other physiological motion. However, the present inventors discovered that a higher bandwidth technique may be realized via the aforementioned NCR measurement system that utilizes a secondary measurement method such as an ECG amplifier with electrode connections to the patient, wherein the ECG amplifier device, for example, modulates the resonant frequency of a near-field coupled resonant antenna that is integrated into the ECG amplifier box or patch. The secondary device is used to take direct physiological measurements, such as ECG. The resonant coil/antenna of the secondary device is coupled to the primary resonant coil (i.e., a primary coil/antenna within the MRI machine). And the impedance of the secondary resonant coil (i.e., the resonant coil/antenna of the secondary device) is modulated with physiological signal(s) to affect the S11 reflection measurement of the primary coil.

The present inventors further realized that detecting physiological signals, such as ECG, wirelessly, with extremely high bandwidth and low power consumption, for gating or synchronization of an MRI scan with patient physiology, in an MRI environment is extremely difficult, requiring extraordinarily high bandwidth to allow the filtering/compensation of MR interference such as RF pulses and changing gradients and requiring immunity to high magnetic and RF fields, while avoiding introducing RF interference for an MRI scan.

Accordingly, the present inventors have developed the disclosed method (and system therefor) of wireless physiological gating in a magnetic resonance imaging (MRI) machine that comprises wirelessly receiving an analog physiological signal of interest from a secondary device via a reflectometer circuit and an antenna of the MRI machine, the antenna of the MRI machine being coupled in near-field with a resonant antenna of the secondary device, and the secondary device having circuitry configured to vary an impedance of or otherwise vary a tuning of the resonant antenna of the secondary device so as to influence an electromagnetic environment and a tuning of the antenna of the MRI machine, the change in tuning of the antenna of the MRI machine being detected by the reflectometer circuit within the MRI machine and corresponding to the analog physiological signal of interest from the secondary device.

The present inventors determined the disclosed techniques provide realtime ECG with high fidelity and bandwidth, with low cost and low power consumption, that will allow high-quality physiological gating signals with immunity to MRI scanner interference. The disclosed techniques also provide considerably less latency (via a directly modulated signal) as opposed to a wireless protocol like Bluetooth or Wi-Fi, leading to improved time-accuracy for gating, resulting in cleaner MR images. Further, existing wireless ECG devices emit RF and, thus, require regulatory approval, whereas with the disclosed techniques, the wireless ECG device does not emit RF and, thus, does not require regulatory approval. Further still, the disclosed techniques utilize electrically modulating the impedance of the resonant antenna of the secondary device (as opposed to mechanically altering a physical/structural shape of the antenna), does not require the use of discrete switches to change the impedance of the secondary coil (i.e., resonant antenna of the secondary device), does not operate by measuring phase, and utilizes near-field coupling (as opposed to RF backscatter).

The present inventors have recognized that no known prior art or existing physiological gating systems in an MRI use impedance modulation or near-field interaction as a way to bring an analog physiological signal from a secondary device, no known MRI accessories make use of “non-contact” (e.g., NCR) devices to receive information or signals from other secondary devices, and no known MRI accessories transmit data in forms other than digital modes or protocols such as Bluetooth or Wifi.

In view of the foregoing, the present inventors developed the disclosed techniques, which may, in addition to the previously described advantages, be significantly less expensive than existing solutions and provide significantly higher bandwidth than existing products (allowing removal of scan interference artifacts and enabling more accurate triggering and gating).

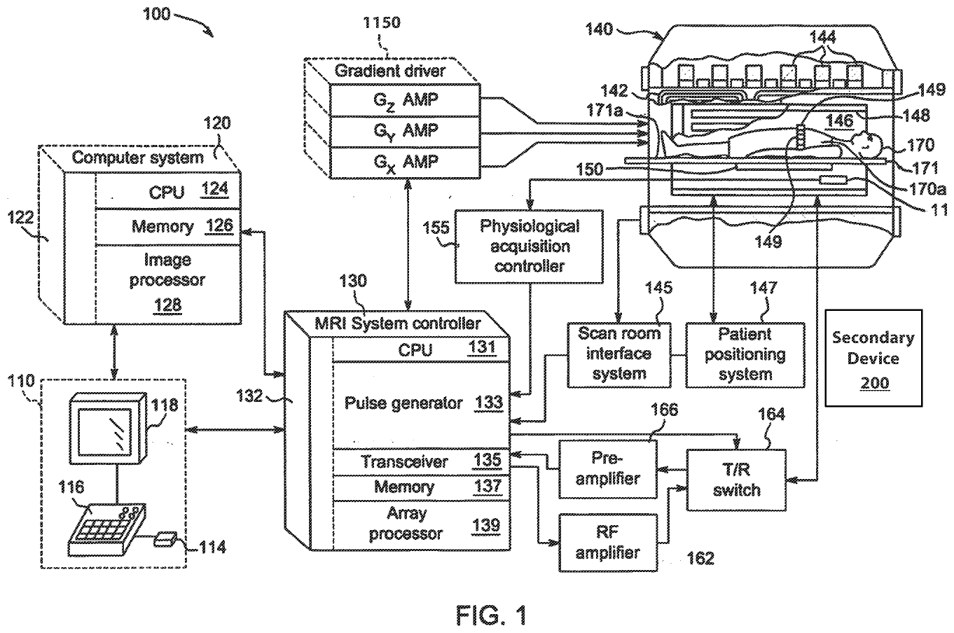

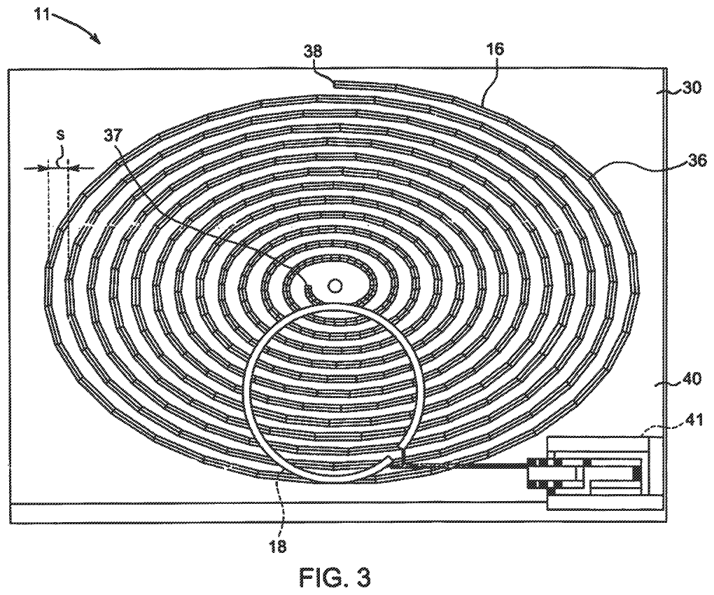

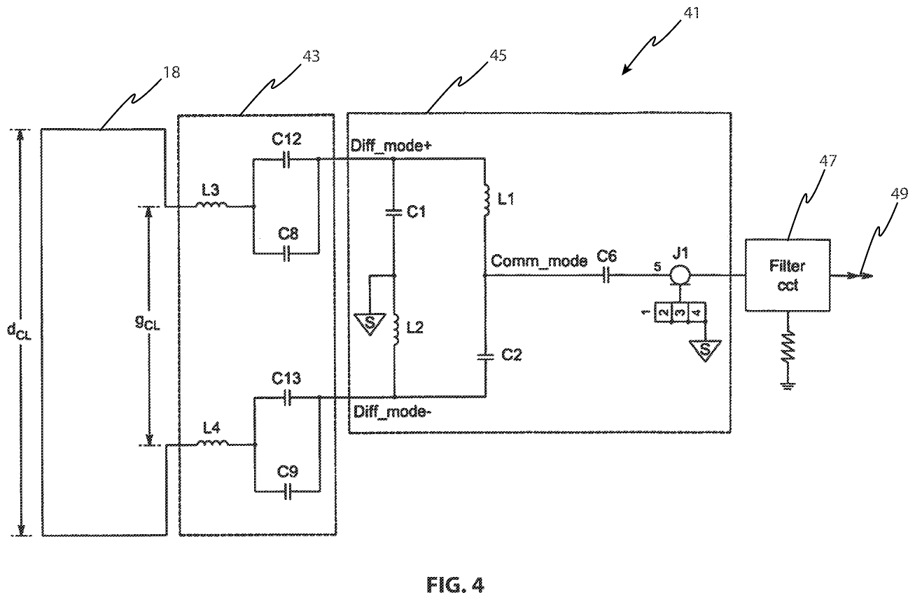

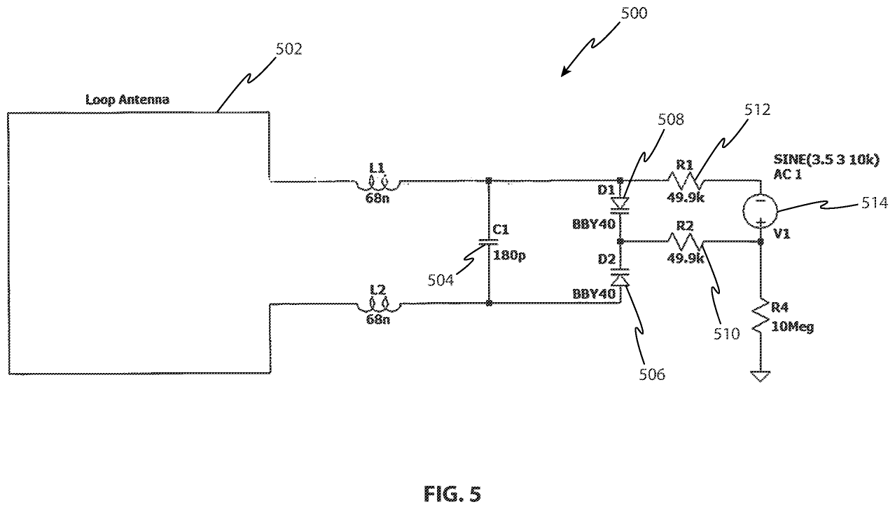

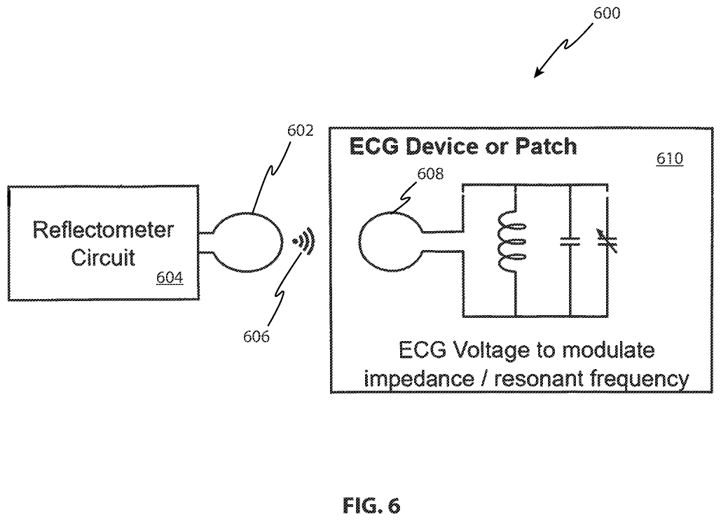

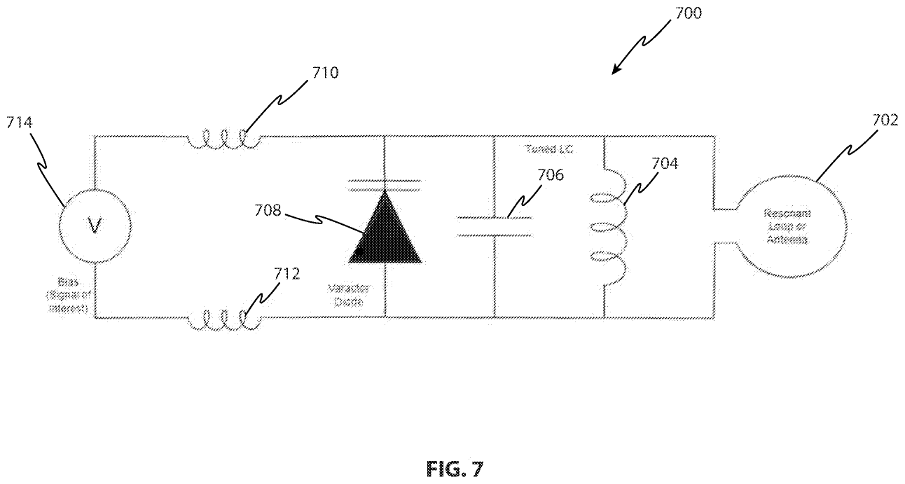

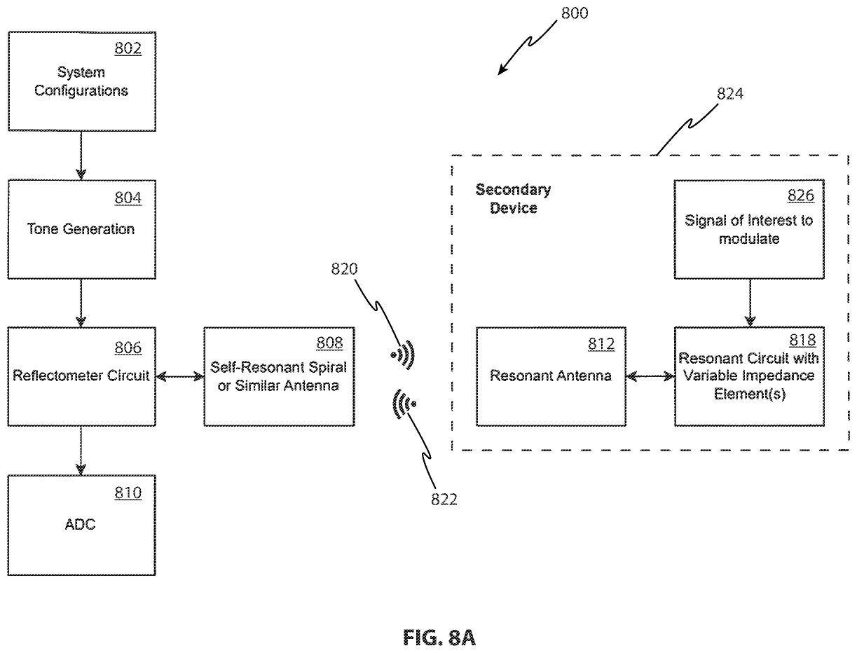

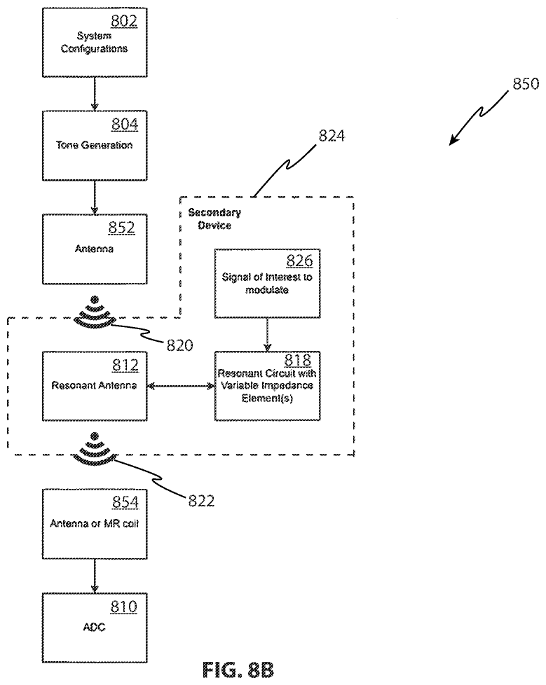

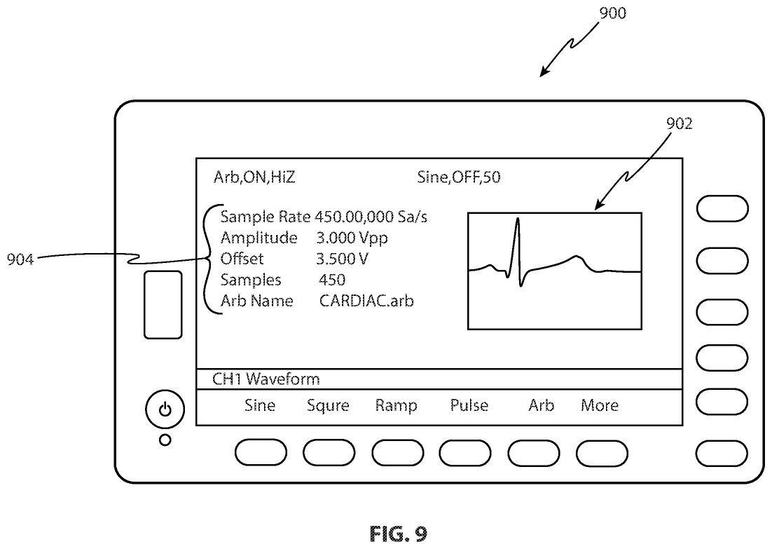

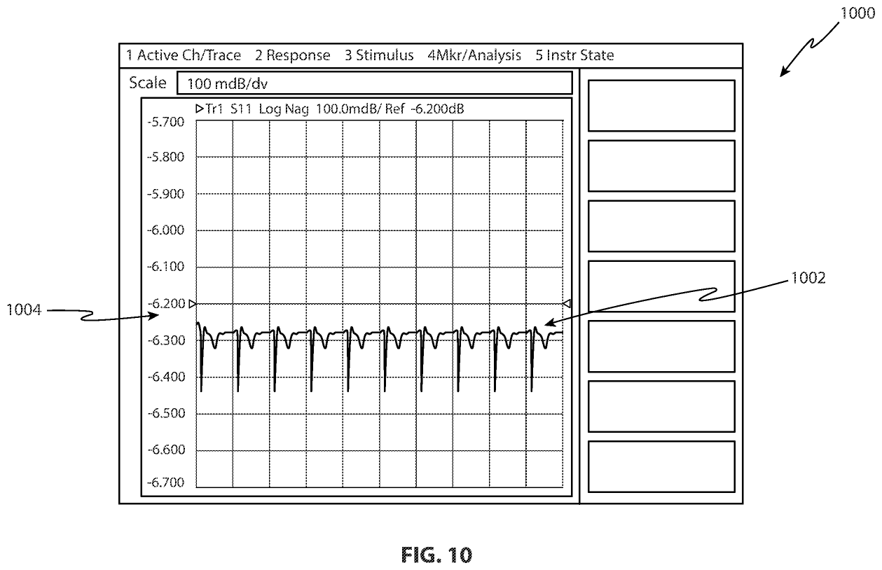

As an overview, FIGS. 1-4 describe an MRI system comprising an MRI machine of the above referenced NCR type MRI system. FIG. 5 illustrates a resonant antenna with variable impedance elements for use in a secondary device (such as an ECG device or patch), comprising modified NCR coupling circuit, that, when driven by a waveform generator and near-field coupled with the NCR MRI machine primary coil/antenna and circuitry of FIGS. 3-4, provides the QRS ECG waveform and plot of changes in S11 in FIGS. 9 and 10, respectively. FIG. 7 depicts a circuit diagram of an exemplary resonant circuit of a secondary device having a varactor diode biased by a signal of interest, to vary an impedance of or otherwise vary a tuning of the resonant antenna of the secondary device. And FIGS. 6 and 8A illustrate a near-field coupling between a primary coil and reflectometer circuit within the MRI machine and a resonant loop or antenna of the secondary device (e.g., ECG device).

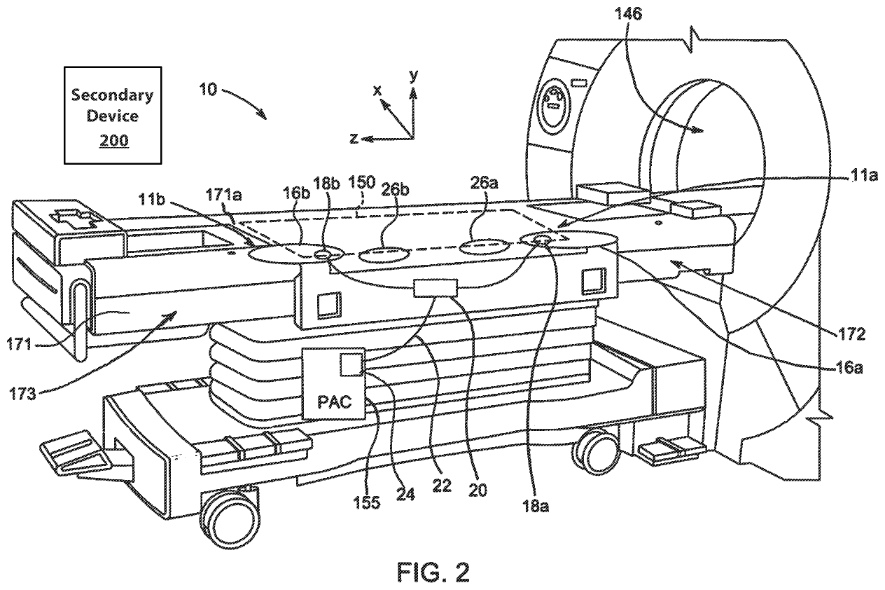

Turning now to the figures, FIG. 1 is a schematic diagram of an exemplary magnetic resonance imaging (MRI) system 100 with an MRI machine and a secondary device 200, according to embodiments. The MRI (or MR imaging) machine generally comprises all of the system components shown in FIG. 1 (and in FIG. 2) (e.g., table 171, resonance assembly 140, computer system 120, MRI system controller 130, physiological acquisition controller (PAC) 155, gradient driver 1150, sensor/motion sensor/primary coil/antenna 11, operator workstation 110, etc.) except for the secondary device 200, and the secondary device 200 is described below in further detail with respect to FIGS. 5-8.

The operation of MRI system 100 is controlled from an operator workstation 110 that includes an input device 114, a control panel 116, and a display 118. The input device 114 may be a joystick, keyboard, mouse, track ball, touch activated screen, voice control, or any similar or equivalent input device. The control panel 116 may include a keyboard, touch activated screen, voice control, buttons, sliders, or any similar or equivalent control device. The operator workstation 110 is coupled to and communicates with a computer system 120 that enables an operator to control the production and viewing of images on display 118. The computer system 120 includes a plurality of components that communicate with each other via electrical and/or data connections 122. The computer system connections 122 may be direct wired connections, fiber optic connections, wireless communication links, or the like. The components of the computer system 120 include a central processing unit (CPU) 124, a memory 126, which may include a frame buffer for storing image data, and an image processor 128. In an alternative embodiment, the image processor 128 may be replaced by image processing functionality implemented in the CPU 124. The computer system 120 may be connected to archival media devices, permanent or back-up memory storage, or a network. The computer system 120 is coupled to and communicates with a separate MRI system controller 130.

The MRI system controller 130 includes a set of components in communication with each other via electrical and/or data connections 132. The MRI system controller connections 132 may be direct wired connections, fiber optic connections, wireless communication links, or the like. The components of the MRI system controller 130 include a CPU 131, a pulse generator 133, which is coupled to and communicates with the operator workstation 110, a transceiver 135, a memory 137, and an array processor 139. In an alternative embodiment, the pulse generator 133 may be integrated into a resonance assembly 140 of the MRI system 100. The MRI system controller 130 is coupled to and receives commands from the operator workstation 110 to indicate the MRI scan sequence to be performed during a MRI scan. The MRI system controller 130 is also coupled to and communicates with a gradient driver system 1150, which is coupled to a gradient coil assembly 142 to produce magnetic field gradients during a MRI scan.

The pulse generator 133 may also receive data from a physiological acquisition controller (PAC) 155 that receives signals from a plurality of different sensors connected to an object or patient 170 undergoing a MRI scan, including respiration signals and/or cardiac signals (e.g., ECG signals). And finally, the pulse generator 133 is coupled to and communicates with a scan room interface system 145, which receives signals from various sensors associated with the condition of the resonance assembly 140. The scan room interface system 145 is also coupled to and communicates with a patient positioning system 147, which sends and receives signals to control movement of a table 171. The table 171 is controllable to move the patient in and out of the bore 146 and to move the patient to a desired position within the bore 146 for a MRI scan.

The MRI system controller 130 provides gradient waveforms to the gradient driver system 1150, which includes, among others, G_{X}, G_{Y} and G_{Z} amplifiers. Each G_{X}, G_{Y} and G_{Z} gradient amplifier excites a corresponding gradient coil in the gradient coil assembly 142 to produce magnetic field gradients used for spatially encoding MR signals during a MRI scan. The gradient coil assembly 142 is included within the resonance assembly 140, which also includes a superconducting magnet having superconducting coils 144, which in operation, provides a homogenous longitudinal magnetic field B₀ throughout a bore 146, or open cylindrical imaging volume, that is enclosed by the resonance assembly 140. The resonance assembly 140 also includes a RF body coil 148 which in operation, provides a transverse magnetic field B₁ that is generally perpendicular to B₀ throughout the bore 146. The resonance assembly 140 may also include RF surface coils 149 used for imaging different anatomies of a patient undergoing a MRI scan. The RF body coil 148 and RF surface coils 149 may be configured to operate in a transmit and receive mode, transmit mode, or receive mode.

An object or patient 170 undergoing a MRI scan may be positioned within the bore 146 of the resonance assembly 140. The transceiver 135 in the MRI system controller 130 produces RF excitation pulses that are amplified by an RF amplifier 162 and provided to the RF body coil 148 and RF surface coils 149 through a transmit/receive switch (T/R switch) 164.

As mentioned above, RF body coil 148 and RF surface coils 149, and/or one or more phased-array (PA) coils 150, may be used to transmit RF excitation pulses and/or to receive resulting MR signals from a patient undergoing a MRI scan. For example, the PA coil(s) 150 may be located in the table underneath the patient 170, such as in an area under the torso 170 a of the patient. The resulting MR signals emitted by excited nuclei in the patient undergoing a MRI scan may be sensed and received by the RF body coil 148, RF surface coils 149, or PA coil 150. Each of the coils 148, 149, and 150 usually include a respective T/R switch, and each usually include the T/R function and preamps within the surface coil/PA coil itself. Thus multiple T/R switches are included in the system, which are collectively represented as T/R switch 164. Similarly, multiple preamps may be included, which are collectively represented as pre-amplifier 166. The amplified MR signals are demodulated, filtered and digitized in the receiver section of the transceiver 135. The appropriate T/R switch 164 is controlled by a signal from the pulse generator 133 to electrically connect the amplifier 162 to the appropriate coil 148, 149, 150 during the transmit mode and connect the corresponding pre-amplifier 166 to the coil 148, 149, 150 during the receive mode. The resulting MR signals sensed and received by the RF body coil 148 or the PA coil 150 are digitized by the transceiver 135 and transferred to the memory 137 in the MRI system controller 130.

A MR scan is complete when an array of raw k-space data, corresponding to the received MR signals, has been acquired and stored temporarily in the memory 137 until the data is subsequently transformed to create images. This raw k-space data is rearranged into separate k-space data arrays for each image to be reconstructed, and each of these separate k-space data arrays is input to the array processor 139, which operates to Fourier transform the data into arrays of image data.

The array processor 139 uses a known transformation method, most commonly a Fourier transform, to create images from the received MR signals. These images are communicated to the computer system 120 where they are stored in memory 126. In response to commands received from the operator workstation 110, the image data may be archived in long-term storage or it may be further processed by the image processor 128 and conveyed to the operator workstation 110 for presentation on the display 118. In various embodiments, the components of computer system 120 and MRI system controller 130 may be implemented on the same computer system or a plurality of computer systems.

A sensor or motion sensor 11 (also comprising what is referred to herein as a primary coil or antenna of the MRI machine) is integrated into the resonance assembly 140 to sense motion of the patient and/or to probe the electromagnetic environment and measure changes in a reflection coefficient corresponding to an analog physiological signal of interest from the secondary device 200. The detected motion information and/or the analog physiological signal of interest (e.g., an ECG or cardiac signal) can be utilized for controlling and optimizing imaging, such as for aiding the MR image capture based on detected periodic motion and/or otherwise improving image quality by avoiding image degradation due to patient motion and/or, for example, patient ECG. The sensor/motion sensor 11 generates a magnetic field by which motion of the patient and/or other physiological signal(s) can be detected, as described herein below. The motion/physiological information is provided to the physiological acquisition controller (PAC) 155, which provides information about the periodic and/or other motion of the patient to the pulse generator 133. For example, the PAC controller 155 may generate a respiration signal and/or an ECG signal formatted for use in triggering MR image data acquisition performed by the MRI system controller 130.

FIG. 2 is an illustration of an exemplary table of an MRI system/machine, according to embodiments. The table 171 is shown schematically illustrating an exemplary sensor/motion sensor system 10 incorporated therein. The table 171 is outside of the bore 146 and is moveable into the bore 146 in order to conduct patient imaging. The table 171 has a top surface 171a for supporting the patient 170 being imaged. In the depicted example, the table includes a PA coil 150 positioned below the top surface 171a. In the depicted example, two sensors/motion sensors 11a and 11b are located below the PA coil and are configured to sense motion of the patient and/or to wirelessly receive a physiological signal of interest from the secondary device 200 (e.g., an ECG signal). In other embodiments, the sensor/motion sensor 11a, 11b may be located elsewhere in the system 140, such as directly below the top surface 171a or elsewhere with respect to the patient, such as on the sides or above the patient in the bore 146.

Each sensor/motion sensor 11a, 11b may include a resonant coil 16a, 16b and a corresponding coupling loop 18a, 18b. Each coupling loop 18a, 18b is configured to generate a drive RF signal to excite the corresponding resonant coil 16a, 16b to radiate a magnetic field having a predefined resonant frequency. The coupling loop 18a, 18b is further configured to receive a reflection RF signal from the corresponding resonant coil 16a, 16b.

In other embodiments, differing drive methods may be utilized, such as via a directly connected driver/receiver. In the case of a direct drive configuration, the sensing of S11 would also be accomplished through the direct drive connection. In one such embodiment, the self-resonant spiral (SRS) coil 36 may consist of two spiral elements interleaved with one rotated 180 degrees from the other. Each interleaved spiral element has a center end and an outer end. The ends closest to the center of the coil 36 may be driven directly using a voltage source in order to excite the self-resonant spiral (SRS) coil to generate the magnetic field. The receipt of the RF signal and sensing of S11 therefrom would also be accomplished through the direct connection. Thus, a coupling loop may be eliminated in a direct drive embodiment.

Each sensor/motion sensor 11a, 11b may be located such that a relevant portion of the patient 170 is within a region of strong magnetic field with respect to the sensor 11a, 11b. Where respiration motion is being detected by the motion sensor 11a, 11b, the sensor/motion sensor 11a, 11b may be positioned such that at least a portion of the torso 170a of the patient 170 is within an area of sufficiently strong magnetic field such that the motion of the torso 170a due to respiration can be detected. Time-varying loading of the magnetic field, the H-field, caused by the changes in absorption of the patient's tissue within the field can be measured and correspond to the respiratory cycle.

In one embodiment, this change is detected by measuring the reflection coefficient (S11) of the RF source power emitted by the coupling loop 18a, 18b into the resonant coil 16a, 16b. The reflection coefficient S11 represents how much power is reflected from the resonant coil 16a, 16b, which will be impacted by the changes in absorption by the patient due to respiration. Accordingly, a respiration signal can be determined based on changes in the reflection coefficient over a respiration period.

In the embodiment at FIG. 2, two sensors/motion sensors 11a, 11b are included. In other embodiments, only one sensor/motion sensor 11 may be included, or more than two sensors/motion sensors 11 may be included. One or more of the sensors/motion sensors 11a, 11b are selectable via a switch 20. In the depicted example, only one of the sensors/motion sensors 11a or 11b are selectable by connecting the respective coupling loop 18a, 18b to the controller, which in the example is the PAC controller 155. The appropriate sensor/motion sensor 11a or 11b is selected based on the direction of the patient for imaging i.e. whether the patient is positioned head-first or feet-first for moving into the bore 146. In the example, sensor/motion sensor 11a is positioned closer to the front end 172 of the table 171 (the end that enters the bore first) and the sensor/motion sensor 11b is positioned closer to the back end 173 of the table 171 (the end that enters the bore last). The sensor/motion sensor 11a will be utilized if the patient is positioned head-first, where the patient's head is at the front end 172 of the table 171. Specifically, the sensor/motion sensor 11a is located so that it will align with the patient's torso 170 a when the patient is positioned head-first toward the bore 146. Alternatively, if the patient is positioned feet-first toward the bore 146, then the sensor/motion sensor 11b may be selected via the switch 20, which is located to align with the patient's torso 170 a when the patient is in the feet-first position.

In one example, selection of the appropriate sensor/motion sensor 11a or 11b by the switch 20 may be controlled based on whether the patient 170 to be imaged is positioned head-first or feet-first. The patient position is known, for example, by the MRI system controller 130 and is a parameter used for multiple control purposes within the MRI system 100. In one embodiment, actuation of the switch 20 to control selection of the sensor/motion sensor 11a or 11b may be performed by providing a pre-defined DC bias on the drive signal coax cable, where a different pre-defined DC bias is associated with each sensor/motion sensor 11a and 11b.

The sensors/motion sensors 11a and 11b may be connected to the controller 155, such as via a coax cable 22. The controller 155 may be a PAC controller 155 comprising a respiration and/or physiological signal (e.g., an ECG signal from the secondary device 200) detection sub-controller 24 that includes circuitry for filtering and digitizing the analog reflectometer measurement provided by the sensor/motion sensor 11a, 11b and software for processing the digitized signal in order to generate a respiration and/or physiological signal that can be utilized for controlling MR image acquisition.

Also as shown in FIG. 2, one or more passively coupled elements 26a, 26b may be included and located adjacent to the active coil 16a, 16b in order to increase the size of the magnetic field along the Z-axis (which extends along the length of the table 171 and head-to-toe with respect to the patient). For example, the coupled elements may be passive SRS coils, and thus do not have a corresponding coupling loop, but are inductively coupled by the magnetic field radiated by the driven SRS coil 36.

FIG. 3 depicts an exemplary primary coil/antenna and matching circuitry, or sensor, of an MRI system/machine, according to embodiments. FIG. 3 depicts an embodiment where the resonant coil 16 is a self-resonant spiral (SRS) coil 36. In other embodiments, the resonant coil 16 may instead by a circular coil or an air coil located at closer proximity to the posterior of the patient 170. In one embodiment, the self-resonant spiral provides for a larger H-field generation due to its multi-turn nature with the tuning capacitance dominated by the distributed capacitance between turns of the spiral. This allows the SRS coil 36 to be located further away from the patient than other types of resonant coils, while still providing useful detection of the patient loading of the field that is sufficiently sensitive to provide good detection of patient respiration, for example. The SRS coil 36 with multiple turns is driven at a frequency below that of the proton scanning, or Larmor frequency, to produce a strong H-field with a large depth of penetration into the scanner subject that will not create interference with the MR imaging.

FIG. 3 depicts one embodiment of an SRS coil 36. The exemplary SRS coil 36 includes 13 turns between a first end 37 and a second end 38 of the conductor, or wire, comprising the coil. In the example, the circular coils are consistently spaced apart, with spacing S being another parameter affecting the resonance frequency and the magnitude of the H-field. In other examples, and depending on the application, a different number of turns and/or different spacing may be utilized, and the spacing may vary depending on the shape of the SRS coil 36. For an elliptical coil, for example, the spacing will vary as a function of the angle of rotation around the center of the coil and as a function of the eccentricity of the ellipse. For instance, the inventors have recognized that various numbers of turns, such as between 10 and 15 turns, may be appropriate depending on the desired resonance frequency and the needed H-field magnitude, which may depend on placement within the table, for example. In the example at FIG. 3, the SRS coil 36 is elliptical. In other embodiments, a differing shape may be used. An elliptical self-resonant spiral coil may generate a stronger magnetic field for a given excitation current. Moreover, the elliptical coil may have the added benefit that it will fit in a narrower space, which may be beneficial for fitting the sensor 11 into the crowded space of the table 171.

As shown in FIG. 3, the SRS coil 36 may be disposed on a layer/board 30, which is illustrated (for description purposes in FIG. 3) in transparent, beneath (or in back of) a layer/board 40 comprising a coupling loop 18 and associated coupling circuitry 41. The coupling loop 18 is inductively coupled to the SRS coil 36, or other resonant coil 16. The coupling loop 18 is configured to generate a drive RF signal to excite the SRS coil to radiate a magnetic field at a predefined frequency. In one embodiment, the use of a 27 MHz SRS coil 36 is desirable in that it provides for a large H-field generation due to its multi-turn nature with the tuning capacitance dominated by the distributed capacitance between the turns of the spiral. 27 MHz is beneficially within an Industrial, Scientific, and Medical (ISM) band. In other embodiments, a different predefined resonant frequency may be utilized, which may be a different ISM band frequency. To provide one example, the predefined resonant frequency may be in the ISM band between 26.975 MHz and 27.283 MHz, or may be between 40.66 MHz and 40.7 MHz, or in still other embodiments may be between 13.553 MHz and 13.567 MHz. In other embodiments, the predetermined resonant frequency may be different and/or outside of those ISM bands. In certain examples, it may be beneficial to utilize a predetermined resonant frequency that is below that of the proton scanning frequency.

The coupling loop 18 also receives a reflection RF signal from the SRS coil 36 such that a respiratory, or other patient motion, signal can be detected by measurement of the change in the reflection RF signal due to the variation in loading that the patient presents to this RF H-field. As the patient breathes, for example, the amount of power reflected by the SRS coil 36 will change. In one embodiment, the motion signal, such as the respiration signal, is determined based on a reflection coefficient S11 of the SRS coil 36. In the depicted embodiment at FIG. 4, a dual log power detection integrated circuit is used in combination with directional couplers to measure the reflected power, the reflection RF signal, divided by the forward power, the drive RF signal, delivered at 27 MHz. The reflection coefficient S11 can then be calculated according to the following equation:

S11=log 10(P_{refl})−log 10(P_{drv})

FIG. 4 is a circuit diagram of an exemplary coupling circuit and matching circuitry of an MRI system/machine, according to embodiments. FIG. 4 depicts one embodiment of a coupling board 40, including the coupling loop and the coupling circuit 41 for detecting the patient motion based on changes in the reflection RF signal. The coupling loop 18 may be, for example, a circular loop of a specified diameter d_{cl}. To provide just one example, the diameter d_{cl} of the coupling loop 18 may be 50 mm, and the gap g_{cl} between the ends of the coupling loop 18 may be, for example, 5 mm. The coupling circuit 41 includes a blocking network 43 that blocks other resonant frequencies other than those at 27 MHz, or the predefined resonant frequency. A lattice balun circuit 45 is further included that translates a differential output of the loop to a single-ended coax feed line. The lattice balun is effective at desensitizing the sensor assembly to frequency shifts. A filtering circuit 47, such as a diplexer, is further included to filter the outputted reflectometer measurement signal prior to transmission to the controller 155. The resulting signal is provided to the socket 49 such as a coax connector.

FIG. 5 is a circuit diagram as shown in FIG. 4 with modifications to provide an exemplary resonant antenna and resonant circuit 500 with variable impedance/variable antenna tuning element(s) 506, 508 of a secondary device (such as for a secondary device 200 in the MRI system 100), according to embodiments. That is, the exemplary resonant antenna and resonant circuit 500, as shown in FIG. 5, may comprise a resonant antenna or loop antenna 502 as shown and described with respect to FIGS. 3 and 4, with lumped element balun components removed, two BBY-40 varactor diodes 506, 508 added in series (arranged, as depicted in FIG. 5, cathode to cathode), a pair of 49.9 Kohm resistors 510, 512 arranged as shown to connect a reverse bias waveform input 514 (which may comprise an SMA connector, for example). And an additional 100 pF capacitor may be added, as shown, to set course tuning. The resulting circuitry comprises a resonant antenna and resonant circuit with variable impedance elements, whereby a signal of interest (such as an analog physiological signal, for example, comprising a QRS signal or an ECG waveform) may be introduced at the input 514.

The present inventors determined that reflectometry, near-field coupling, and wirelessly receiving an analog physiological signal such as an analog ECG waveform, by modulating the resonant antenna of a secondary device, to effectively prototype and prove out such characteristics for an MRI system with secondary device as described with respect to FIGS. 1 and 2, may be accomplished using a modified NCR antenna assembly (i.e., modifying the NCR antenna assembly described with respect to FIGS. 3 and 4 so as to comprise the resonant antenna and resonant circuit shown in FIG. 5), and using an a arbitrary waveform generator with adjustable amplitude and offset to modulate the (secondary device) resonant coil/loop antenna 502 tuning. The waveform generator may be configured, for example, to provide 3 VDC bias+/−3V pk-pk analog signal of interest comprising the QRS signal or ECG waveform illustrated in FIG. 9.

The above-described test set up may comprise positioning the resonant coil/loop antenna 502 at a distance of 25 cm from a self-resonant coil 36 and reflectometer circuitry shown and described with respect to FIGS. 3 and 4, so that the resonant antenna 502 is coupled in near-field with the self-resonant coil 36 (or antenna of the primary device/MRI machine). A network analyzer may then be connected with the NCR antenna assembly described with respect to FIGS. 3 and 4, for driving the reflectometer circuitry and measuring changes in the reflection coefficient S11, which may then be displayed, as illustrated in FIG. 10, as S11 measurements, with, for example, 0.2 dB pk-pk S11 variation.

With the above described test set up, the present inventors proved out an embodiment for wirelessly receiving an analog physiological signal of interest (e.g., a QRS signal or ECG waveform) from a secondary device (such as a secondary device 200) via a reflectometer circuit and an antenna of the MRI machine (such as described in FIGS. 1-4), the antenna of the MRI machine (such as described in FIGS. 3 and 4) being coupled in near-field with a resonant antenna (such as resonant or loop antenna 502) of the secondary device, and the secondary device having circuitry configured (such as shown in FIG. 5) to vary an impedance of or otherwise vary a tuning of the resonant antenna 502 of the secondary device 200 so as to influence an electromagnetic environment and a tuning of the antenna 16, 36 of the MRI machine, the change in tuning of the antenna of the MRI machine being detected by the reflectometer circuit within the MRI machine and corresponding to the analog physiological signal of interest (such as the waveform shown in FIG. 9) from the secondary device. And, as described, FIG. 10 illustrates a plot of changes in the reflection coefficient (S11) detected by the reflectometer circuit within the MRI machine, corresponding to the analog physical signal of interest (e.g., QRS signal or ECG waveform) from the secondary device 200.

FIG. 6 is a schematic diagram a system 600 comprising a primary coil/antenna 602 and reflectometer circuit 604 within a primary device (such as an MRI machine as described with respect to FIGS. 1-2) coupled in near-field to an exemplary secondary device 610, according to embodiments. A primary resonant coil 602 may comprise, for example a self-resonant spiral (SRS) or similar antenna (e.g., SRS 36) and is driven by a reflectometer circuit 604 so as to emit (or radiate) a magnetic field 606 having a predefined resonant frequency (or tone). The resonant frequency may be, for example, between 26.957 MHz and 27.283 MHz. The reflectometer circuit 604 may be positioned within the MRI machine and may be configured, alone or together with circuitry and/or a controller (such as PAC 155) to detect and process changes in loading of the magnetic field 606. The operation of the reflectometer circuit 604 and the primary coil 602 may be substantially as described with respect to the MRI machine in FIGS. 1-4.

A tuned secondary resonant coil or antenna 608 (of the ECG device or patch 610) influences the electromagnetic environment including the magnetic field 606. And since the primary 602 and secondary 608 coils or antennas are coupled in the near-field, the tuning of the secondary antenna 608 affects the tuning of the primary antenna 602. This change in tuning can be detected as a change in S11 or reflection coefficient on the primary coil 602, by the reflectometer circuit 604.

The ECG device 610 may comprise a pre-amplifier and/or ECG amplifier box, from which electrodes extend for physical contact and connection to a patient (such as patient 170). The ECG device 610 may comprise a patch or flexible circuit with adhesive or other connection for contact and connection to a patient 170. The ECG device or patch 610 may comprise pads which are specialized and manufactured as disposable devices, with circuitry embedded therein. As indicated FIG. 6, the ECD device or patch 610 may comprise circuitry or contact leads to receive, for example, an ECG voltage and circuitry configured to modulate an impedance or resonant frequency of the secondary coil 608 corresponding to the ECG voltage (which comprises an analog physiological signal of interest).

FIG. 7 is a circuit diagram of a secondary circuit 700 of an exemplary resonant antenna and resonant circuit of a secondary device 200, 610, according to embodiments. The secondary device, for example, may comprise a secondary circuit 700 that includes a resonant loop or antenna (or secondary coil) 702, a tuned LC circuit comprising inductor 704 and capacitor 706 elements/components, and a variable impedance element 708 such as a varactor diode, connected in parallel with one another as shown, and connected via inductors 710 and 712 to a bias or signal of reference (e.g., bias voltage) 714. In operation, changes in the bias voltage 714 cause corresponding changes in the variable impedance element 708, which varies an impedance of the resonant loop/antenna (secondary device antenna) 702. Other configurations of circuitry may be used to modulate an impedance of the secondary device antenna/resonant antenna 702.

For the secondary circuit 700 shown in FIG. 7, a tuning of the secondary antenna is changed by a variable impedance element, controlled by a signal of interest (e.g., bias voltage) 714. In one embodiment, the signal of interest is applied as a bias signal to a varactor diode 708 which changes the capacitance of the secondary circuit 700. This change in capacitance changes the tuning of the secondary coil 702, and by coupling and near-field interaction, changes the tuning of the primary coil (e.g., primary coil 602).

FIG. 8A is a schematic diagram of an MRI system 800 having a self-resonant spiral or similar antenna 808 and a reflectometer circuit 806 of the MRI machine coupled in near-field with a resonant antenna 812 and resonant circuit 818 of a secondary device 824, according to embodiments. As shown in FIG. 8A, the schematic functional blocks positioned/arranged to the left of the radiated magnetic field 820 may be located or may reside within the primary device or MRI machine, and the schematic functional blocks positioned/arranged to the right of the radiated magnetic field 820 may be located or may reside within the secondary device or ECG device.

In various embodiments, the secondary device 824 is separate from the primary device, and the secondary device is free from electrical interconnection with the primary device and/or the secondary device is free from requiring any wired electrical interconnection with the primary device so as to permit data transfer of a physiological signal of interest 826 from the secondary device to the primary device and/or the secondary device is free from requiring using any radio frequency (RF) or other type of emitting or radiating transmitter.

In an embodiment, the primary device comprises an interface for inputting and/or changing system configurations 802. One such input may be selection of a predetermined resonant frequency at which the primary coil or antenna of the primary device is driven or excited so as to radiate (emit) the magnetic field 820. With the predetermined resonant frequency (or frequency tone) selected, circuitry for tone generation 804 (such as, for example, phase locked loop (PLL) or other circuitry) generates a constant frequency tone. Next, a reflectometer circuit 806 of the primary device drives the primary coil or antenna of the primary device (such as, for example, a self-resonant spiral (SRS) or similar antenna 808) to excite the primary coil (e.g., SRS coil) to radiate the magnetic field 820 having the predefined resonant frequency. The primary coil is excited such that incident waves are emitted by the resonant coil/resonant primary antenna 808. In one embodiment, the reflectometer circuit 806 measures the incident and reflected waveform, giving an S11 measurement. In one embodiment, the reflectometer circuit 806 detects and measures a ratio of incident and reflected power, for example, using a log-power-ratio detector, comparing incident and reflected power.

The secondary resonant antenna 812 is coupled to the primary antenna 808 by near-field coupling. Therefore, the impedance and resonant frequency of the primary antenna 808 is dependent on the tuning of the secondary antenna 812. In one embodiment, the secondary resonant antenna 812 has the means to modulate its impedance/tuning/resonant frequency. For example, a varactor or varicap diode may be used to provide a variable capacitance controlled by a reverse bias voltage. This reverse bias can be driven to correlate to a physiological signal of interest 826, such as an ECG signal. As the varicap changes capacitance, the impedance of the coupled system changes. This effectively “transmits” (e.g., via modulating impedance, influencing the electromagnetic environment 822) real-time ECG data from the secondary device 824 to the primary device. In one embodiment, the real-time ECG data is effectively “transmitted” from the secondary device to the primary device and an ADC 810 that measures the ratio of incident and reflected power, or S11 reflection coefficient.

In one embodiment, the exemplary secondary device 824 (e.g., an ECG device) illustrated in FIG. 8A is configured to: generate an analog physiological signal of interest 826 (e.g., ECG) via electrode connections or other connection to a patient (e.g. a patient 170) and a resonant circuit 818 of the secondary device configured to directly modulate an impedance of or otherwise directly modulate the tuning of the resonant antenna 812 of the secondary device so as to correlate to the analog physiological signal of interest; and modulate the impedance of or otherwise modulate the tuning of the resonant antenna 812 of the secondary device so as to correlate to the analog physiological signal of interest and to influence the electromagnetic environment and the tuning of the SRS or similar antenna 808 of the primary device (e.g., the primary device comprising an MRI machine as described in FIGS. 1-4).

Power for the secondary device could be provided by a battery, other means of power transmission such as photovoltaic/solar, MRI RF/Gradient power harvesting, and/or by power harvesting from the NCR primary coil. As previously mentioned, the secondary device may comprise the form factor of a traditional wireless ECG used in conventional MR, such as a box with a battery and ports for connections to ECG leads and other transducers or measurement sensors. Or the form factor may be that of a wireless ECG “patch” with one or more flexible circuit with electrodes or sensors integrated that can be affixed to patient.

Further, while the secondary device 200 is principally described herein in the context of an ECG device, the present disclosure includes aspects applicable to other systems, other modalities, or in other situations. For example, the analog physiological signal of interest may comprise a squeeze bulb that may be triggered/squeezed by the patient 170 in order to gain the attention of, for example, an MRI system operator. Such a squeeze bulb application may involve, for example, resonant circuitry 818 configured to vary an impedance of or otherwise vary a tuning of the (secondary device) resonant antenna to a particular impedance varying frequency and/or wave form and/or pattern.

Also, in other embodiments, different methods of modulation may be used, or different methods of how the modulated signal is detected may be used. For example, while the aforementioned and described MRI system 100 may utilize S11 or reflection coefficient measurements, different methods of detection (via circuitry within the MRI machine) may be used, such as, for example, S21 transmission coefficient, measuring the transmission path, power consumption, detecting resonant frequency, or sensing transmitter current or voltage.

FIG. 8B is a schematic diagram of an MRI system 850 configured to measure a transmission path between a transmit antenna 852 of the primary device (e.g., the MRI machine) and a receive antenna 854 of the primary device, wherein the secondary device 824 may be (near-field) coupled to either the transmit antenna 852 or the receive antenna 854, or both the transmit antenna 852 and the receive antenna 854 (of the primary measurement system). In one embodiment, the real-time physiological signal of interest data (e.g., ECG data) (as described with respect to FIG. 8A) is effectively “transmitted” from the secondary device 824 to the primary device and an ADC 810 that measures the transmission path, or S21 transmission coefficient. In various embodiments, the system configuration 802, tone generation 804, radiated magnetic field 820, modulating impedance/influencing the EM environment 822, and other schematic functional blocks shown in FIG. 8B operate and function as described with respect to FIG. 8A. In one embodiment, the receive antenna 854 may be specifically designed for detection of the modulated EM environment. In another embodiment, the receive antenna 854 may comprise existing MR coils of the MRI machine used for imaging.

Whereas FIG. 8A depicts using reflectometry to probe the electromagnetic (EM) environment (illustrating one implementation using a reflectometer to probe the EM environment and whereby the secondary device modulates the EM environment), FIG. 8B depicts measuring transmission path, for example, using a separate transmit antenna 852 and receive antenna 854 (of the primary device) to probe the EM environment (whereby the secondary device 824 still modulates the EM environment). In further embodiments, other methods may be used to probe the EM environment or probe a secondary device 824.

FIG. 9 depicts a waveform generator display 900 displaying a shape 902 of an exemplary analog physiological signal of interest, according to embodiments. The shape 902 comprises a QRS signal or ECG waveform and is generated as an arbitrary analog waveform for input into a resonant circuit of a secondary device (such as the exemplary resonant antenna and resonant circuit 500 at waveform input 514). Exemplary settings 904, include, as shown: sample rate (450 samples/sec), amplitude (3 Vpp), offset (3.5V), samples (450), arbitrary waveform name (Cardiac.arb).

FIG. 10 depicts a network analyzer display 1000 displaying a plot 1002 of changes in a reflection coefficient (S11) for an exemplary primary coil/antenna and reflectometer circuit, according to embodiments. The vertical axis of the plot 1002 is S11 (dB), and a resonant frequency of the primary antenna of 27 MHz is used.

The present disclosure provides support for a method for wireless physiological gating in a magnetic resonance imaging (MRI) machine, the method comprising: wirelessly receiving an analog physiological signal of interest from a secondary device and a primary device, the primary device comprising an antenna of the MRI machine being coupled in near-field with a resonant antenna of the secondary device, and the secondary device having circuitry configured to vary an impedance of or otherwise vary a tuning of the resonant antenna of the secondary device so as to influence an electromagnetic environment or tuning of the antenna of the MRI machine, the change in electromagnetic environment being detected by the primary device and corresponding to the analog physiological signal of interest from the secondary device. In one aspect, the primary device includes circuitry and one or more antenna of the MRI machine configured to detect the change in electromagnetic environment; or the antenna of the MRI machine comprises a self-resonant spiral or other type of antenna configured to, when electrically driven by a reflectometer circuit of the primary device, probe the electromagnetic environment and measure changes in a reflection coefficient corresponding to the analog physiological signal of interest from the secondary device; or the MRI machine comprises circuitry and a transmit antenna and a receive antenna configured to probe the electromagnetic environment and measure a transmission path and/or changes in a transmission coefficient corresponding to the analog physiological signal of interest from the secondary device. In another aspect, the analog physiological signal of interest comprises an electrical QRS signal or electrocardiogram (ECG) waveform, and the secondary device comprises an ECG device or ECG patch with electrode connections to a patient and a resonant circuit configured to directly modulate an impedance of or otherwise directly modulate the tuning of the resonant antenna of the secondary device so as to correlate to the ECG waveform. In another aspect, the secondary device is free from electrical interconnection with the MRI machine and is configured so as to permit wireless data transfer of the analog physiological signal of interest from the secondary device to the MRI machine without the secondary device using a radio frequency (RF) transmitter. In another aspect, the method includes: generating a constant or varying frequency tone (or signal, which may comprise a “chirp” or “frequency sweep” or similar) using phase locked loop (PLL) or other circuitry, controlled by a system comprising the MRI machine; driving, via a reflectometer circuitry of the MRI machine, the antenna of the MRI machine to emit the constant or varying frequency tone or signal as incident waves emitted by the antenna of the MRI machine; near-field coupling the antenna of the MRI machine and the resonant antenna of the secondary device; and detecting and measuring, by the reflectometer circuit and circuitry associated therewith, a ratio of incident and reflected power, and a reflection coefficient therefor, the changes in the reflection coefficient corresponding to the analog physiological signal of interest from the secondary device. In another aspect, the method includes: generating the analog physiological signal of interest via electrode connections or other connection to a patient and a resonant circuit of the secondary device configured to directly modulate an impedance of or otherwise directly modulate the tuning of the resonant antenna of the secondary device so as to correlate to the analog physiological signal of interest; and modulating the impedance of or otherwise modulating the tuning of the resonant antenna of the secondary device so as to correlate to the analog physiological signal of interest and to influence the electromagnetic environment and the tuning of the antenna of the MRI machine. In another aspect, modulating the impedance of or otherwise modulating the tuning of the resonant antenna of the secondary device comprises biasing a variable impedance circuit element or a varactor or varicap diode of the resonant circuit of the secondary device to provide a variable capacitance that is controlled by a reverse bias voltage, the reverse bias voltage being driven to correlate to the physiological signal of interest. In another aspect, the physiological signal of interest is an electrical QRS signal or electrocardiogram (ECG) waveform or other physiological signal generated via electrode connections or other connection to the patient. In another aspect, the analog physiological signal of interest comprises a signal generated in response to a squeeze bulb that is triggered by a patient squeezing the squeeze bulb. In yet another aspect, the secondary device is powered by a battery and/or photovoltaic cell/solar and/or power harvesting from RF/gradient power from the MRI machine and/or power harvesting from the antenna/primary coil of the MRI machine.

The present disclosure also provides support for a system configured to wirelessly transfer data from a secondary device to a primary device, the system comprising: circuitry and an antenna/primary coil of the primary device configured to wirelessly receive an analog physiological signal of interest from the secondary device, the antenna of the primary device being coupled in near-field with a resonant antenna of the secondary device, and the secondary device having circuitry configured to vary an impedance of or otherwise vary a tuning of the resonant antenna of the secondary device so as to influence an electromagnetic environment or a tuning of the antenna of the primary device, the change in electromagnetic environment being detected by the circuitry within the primary device and corresponding to the analog physiological signal of interest from the secondary device. In one aspect, the primary device includes circuitry and one or more antenna configured to detect the change in electromagnetic environment; or the antenna of the primary device comprises a self-resonant spiral or other type of antenna configured to, when electrically driven by a reflectometer circuit, probe the electromagnetic environment and measure changes in a reflection coefficient corresponding to the analog physiological signal of interest from the secondary device; or the primary device comprises circuitry and a transmit antenna and a receive antenna configured to probe the electromagnetic environment and measure a transmission path and/or changes in a transmission coefficient corresponding to the analog physiological signal of interest from the secondary device. In another aspect, the primary device comprises a magnetic resonance imaging (MRI) machine, wherein the analog physiological signal of interest comprises an electrical QRS signal or electrocardiogram (ECG) waveform, and wherein the secondary device comprises an ECG device or ECG patch with electrode connections to a patient and a resonant circuit configured to directly modulate an impedance of or otherwise directly modulate the tuning of the resonant antenna of the secondary device so as to correlate to the ECG waveform. In another aspect, the secondary device is free from electrical interconnection with the primary device and is configured so as to permit wireless data transfer of the analog physiological signal of interest from the secondary device to the primary device without the secondary device using a radio frequency (RF) transmitter. In still another aspect, the primary device is configured to: generate a constant or varying frequency tone or signal using phase locked loop (PLL) or other circuitry, controlled by a system comprising the primary device; drive, via a reflectometer circuitry of the primary device, the antenna of the primary device to emit the constant or varying frequency tone or signal as incident waves emitted by the antenna of the primary device; near-field couple the antenna of the primary device and the resonant antenna of the secondary device; and detect and measure, by the reflectometer circuit and circuitry associated therewith, a ratio of incident and reflected power, and a reflection coefficient therefor, the changes in the reflection coefficient corresponding to the analog physiological signal of interest from the secondary device. In another aspect, the secondary device is configured to: generate the analog physiological signal of interest via electrode connections or other connection to a patient and a resonant circuit of the secondary device configured to directly modulate an impedance of or otherwise directly modulate the tuning of the resonant antenna of the secondary device so as to correlate to the analog physiological signal of interest; and modulate the impedance of or otherwise modulate the tuning of the resonant antenna of the secondary device so as to correlate to the analog physiological signal of interest and to influence the electromagnetic environment and the tuning of the antenna of the primary device. In yet another aspect, modulating the impedance of or otherwise modulating the tuning of the resonant antenna of the secondary device comprises biasing a variable impedance circuit element or a varactor or varicap diode of the resonant circuit of the secondary device to provide a variable capacitance that is controlled by a reverse bias voltage, the reverse bias voltage being driven to correlate to the physiological signal of interest.

The present disclosure further provides support for a system to wirelessly transfer data from an electrocardiogram (ECG) device to a magnetic resonance imaging (MRI) machine, the system comprising: circuitry and an antenna/primary coil of the MRI machine configured to wirelessly receive an analog physiological signal of interest from the ECG device, the antenna of the MRI machine being coupled in near-field with a resonant antenna of the ECG device, and the ECG device having circuitry configured to vary an impedance of or otherwise vary a tuning of the resonant antenna of the ECG device so as to influence an electromagnetic environment or a tuning of the antenna of the MRI machine, the change in electromagnetic environment being detected by the circuitry within the MRI machine and corresponding to the analog physiological signal of interest from the ECG device, wherein the MRI machine comprises circuitry and one or more antenna configured to probe the electromagnetic environment and measure changes in the electromagnetic environment corresponding to the analog physiological signal of interest from the ECG device. In one aspect, the analog physiological signal of interest comprises an electrical QRS signal or electrocardiogram (ECG) waveform, and wherein the ECG device comprises electrode connections to a patient and a resonant circuit configured to directly modulate an impedance of or otherwise directly modulate the tuning of the resonant antenna of the secondary device so as to correlate to the ECG waveform. In another aspect, the ECG device is free from electrical interconnection with the MRI machine and is configured so as to permit wireless data transfer of the analog physiological signal of interest from the ECG device to the MRI machine without the ECG device using a radio frequency (RF) transmitter. In yet another aspect, the ECG device is powered by a battery and/or photovoltaic cell/solar and/or power harvesting from RF/gradient power from the MRI machine and/or power harvesting from the antenna/primary coil of the MRI machine.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

FIGS. 1-8, or portions thereof, may show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method of wireless physiological gating in a magnetic resonance imaging (MRI) machine, the method comprising:
wirelessly receiving an analog physiological signal of interest from a primary device and a secondary device, the primary device comprising an antenna of the MRI machine being coupled in near-field with a resonant antenna of the secondary device, and the secondary device having circuitry configured to vary an impedance of or otherwise vary a tuning of the resonant antenna of the secondary device so as to influence an electromagnetic environment or tuning of the antenna of the MRI machine, the change in electromagnetic environment being detected by the primary device and corresponding to the analog physiological signal of interest from the secondary device.

2. The method of claim 1, wherein
the primary device includes circuitry and the antenna of the MRI machine configured to detect the change in electromagnetic environment; or
the antenna of the MRI machine comprises a self-resonant spiral or other type of antenna configured to, when electrically driven by a reflectometer circuit of the primary device, probe the electromagnetic environment and measure changes in a reflection coefficient corresponding to the analog physiological signal of interest from the secondary device; or
the MRI machine comprises circuitry and a transmit antenna and a receive antenna configured to probe the electromagnetic environment and measure a transmission path and/or changes in a transmission coefficient corresponding to the analog physiological signal of interest from the secondary device.

3. The method of claim 1, wherein the analog physiological signal of interest comprises an electrical QRS signal or electrocardiogram (ECG) waveform, and wherein the secondary device comprises an ECG device or ECG patch with electrode connections to a patient and a resonant circuit configured to directly modulate an impedance of or otherwise directly modulate the tuning of the resonant antenna of the secondary device so as to correlate to the ECG waveform.

4. The method of claim 1, wherein the secondary device is free from electrical interconnection with the MRI machine and is configured so as to permit wireless data transfer of the analog physiological signal of interest from the secondary device to the MRI machine without the secondary device using a radio frequency (RF) transmitter.

5. The method of claim 1, comprising:
generating a constant or varying frequency tone or signal using phase locked loop (PLL) or other circuitry, controlled by a system comprising the MRI machine;
driving, via a reflectometer circuitry of the MRI machine, the antenna of the MRI machine to emit the constant or varying frequency tone or signal as incident waves emitted by the antenna of the MRI machine;
near-field coupling the antenna of the MRI machine and the resonant antenna of the secondary device; and
detecting and measuring, by the reflectometer circuit and circuitry associated therewith, a ratio of incident and reflected power, and a reflection coefficient therefor, the changes in the reflection coefficient corresponding to the analog physiological signal of interest from the secondary device.

6. The method of claim 5, comprising:
generating the analog physiological signal of interest via electrode connections or other connection to a patient and a resonant circuit of the secondary device configured to directly modulate an impedance of or otherwise directly modulate the tuning of the resonant antenna of the secondary device so as to correlate to the analog physiological signal of interest; and
modulating the impedance of or otherwise modulating the tuning of the resonant antenna of the secondary device so as to correlate to the analog physiological signal of interest and to influence the electromagnetic environment and the tuning of the antenna of the MRI machine.

7. The method of claim 6, wherein modulating the impedance of or otherwise modulating the tuning of the resonant antenna of the secondary device comprises biasing a variable impedance circuit element or a varactor or varicap diode of the resonant circuit of the secondary device to provide a variable capacitance that is controlled by a reverse bias voltage, the reverse bias voltage being driven to correlate to the physiological signal of interest.

8. The method of claim 1, wherein the analog physiological signal of interest comprises a signal generated in response to a squeeze bulb that is triggered by a patient squeezing the squeeze bulb.

9. The method of claim 1, wherein the secondary device is powered by a battery and/or photovoltaic cell/solar and/or power harvesting from RF/gradient power from the MRI machine and/or power harvesting from the antenna/primary coil of the MRI machine.

10. A system configured to wirelessly transfer data from a secondary device to a primary device, the system comprising:
circuitry and an antenna/primary coil of the primary device configured to wirelessly receive an analog physiological signal of interest from the secondary device, the antenna of the primary device being coupled in near-field with a resonant antenna of the secondary device, and the secondary device having circuitry configured to vary an impedance of or otherwise vary a tuning of the resonant antenna of the secondary device so as to influence an electromagnetic environment or tuning of the antenna of the primary device, the change in electromagnetic environment being detected by the circuitry within the primary device and corresponding to the analog physiological signal of interest from the secondary device.

11. The system of claim 10, wherein
the primary device includes circuitry and one or more antenna configured to detect the change in electromagnetic environment; or
the antenna of the primary device comprises a self-resonant spiral or other type of antenna configured to, when electrically driven by a reflectometer circuit, probe the electromagnetic environment and measure changes in a reflection coefficient corresponding to the analog physiological signal of interest from the secondary device; or
the primary device comprises circuitry and a transmit antenna and a receive antenna configured to probe the electromagnetic environment and measure a transmission path and/or changes in a transmission coefficient corresponding to the analog physiological signal of interest from the secondary device.

12. The system of claim 10, wherein the primary device comprises a magnetic resonance imaging (MRI) machine, wherein the analog physiological signal of interest comprises an electrical QRS signal or electrocardiogram (ECG) waveform, and wherein the secondary device comprises an ECG device or ECG patch with electrode connections to a patient and a resonant circuit configured to directly modulate an impedance of or otherwise directly modulate the tuning of the resonant antenna of the secondary device so as to correlate to the ECG waveform.

13. The system of claim 10, wherein the secondary device is free from electrical interconnection with the primary device and is configured so as to permit wireless data transfer of the analog physiological signal of interest from the secondary device to the primary device without the secondary device using a radio frequency (RF) transmitter.

14. The system of claim 10, wherein the primary device is configured to:
generate a constant or varying frequency tone or signal using phase locked loop (PLL) or other circuitry, controlled by a system comprising the primary device;
drive, via a reflectometer circuitry of the primary device, the antenna of the primary device to emit the constant or varying frequency tone or signal as incident waves emitted by the antenna of the primary device;
near-field couple the antenna of the primary device and the resonant antenna of the secondary device; and
detect and measure, by the reflectometer circuit and circuitry associated therewith, a ratio of incident and reflected power, and a reflection coefficient therefor, the changes in the reflection coefficient corresponding to the analog physiological signal of interest from the secondary device.

15. The system of claim 14, wherein the secondary device is configured to:
generate the analog physiological signal of interest via electrode connections or other connection to a patient and a resonant circuit of the secondary device configured to directly modulate an impedance of or otherwise directly modulate the tuning of the resonant antenna of the secondary device so as to correlate to the analog physiological signal of interest; and
modulate the impedance of or otherwise modulate the tuning of the resonant antenna of the secondary device so as to correlate to the analog physiological signal of interest and to influence the electromagnetic environment and the tuning of the antenna of the primary device.

16. The method of claim 15, wherein modulating the impedance of or otherwise modulating the tuning of the resonant antenna of the secondary device comprises biasing a variable impedance circuit element or a varactor or varicap diode of the resonant circuit of the secondary device to provide a variable capacitance that is controlled by a reverse bias voltage, the reverse bias voltage being driven to correlate to the physiological signal of interest.

17. A system to wirelessly transfer data from an electrocardiogram (ECG) device to a magnetic resonance imaging (MRI) machine, the system comprising:
circuitry and an antenna/primary coil of the MRI machine configured to wirelessly receive an analog physiological signal of interest from the ECG device, the antenna of the MRI machine being coupled in near-field with a resonant antenna of the ECG device, and the ECG device having circuitry configured to vary an impedance of or otherwise vary a tuning of the resonant antenna of the ECG device so as to influence an electromagnetic environment or a tuning of the antenna of the MRI machine, the change in electromagnetic environment being detected by the circuitry within the MRI machine and corresponding to the analog physiological signal of interest from the ECG device, wherein the MRI machine comprises circuitry and one or more antenna configured to probe the electromagnetic environment and measure changes in the electromagnetic environment corresponding to the analog physiological signal of interest from the ECG device.

18. The system of claim 17, wherein the analog physiological signal of interest comprises an electrical QRS signal or electrocardiogram (ECG) waveform, and wherein the ECG device comprises electrode connections to a patient and a resonant circuit configured to directly modulate an impedance of or otherwise directly modulate the tuning of the resonant antenna of the secondary device so as to correlate to the ECG waveform.

19. The system of claim 18, wherein the ECG device is free from electrical interconnection with the MRI machine and is configured so as to permit wireless data transfer of the analog physiological signal of interest from the ECG device to the MRI machine without the ECG device using a radio frequency (RF) transmitter.

20. The method of claim 19, wherein the ECG device is powered by a battery and/or photovoltaic cell/solar and/or power harvesting from RF/gradient power from the MRI machine and/or power harvesting from the antenna/primary coil of the MRI machine.
